(19) United States
(12) Patent Application Publication
(10) Pub. No.: US 2010/0010278 A1
(43) Pub. Date: Jan. 14, 2010

(54) PROCESS FOR SELECTIVE HYDROGENATION ON A GOLD-CONTAINING CATALYST

(75) Inventors: Christophe Boyer, Charly (FR); Vincent Coupard, Valencin (FR); Laurent Delannoy, Paris (FR); Antoine Hugon, Lyon (FR); Lars Fischer, Vienne (FR); Catherine Louis, Paris (FR); Cecile Thomazeau, Brignais (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: IFP, Rueil-Malmaison Cedex (FR)

(21) Appl. No.: 12/480,570

(22) Filed: Jun. 8, 2009

(30) Foreign Application Priority Data
Jun. 6, 2008 (FR) 08/03.177

(51) Int. Cl. C07C 5/09

(52) U.S. Cl. 585/260; 585/277; 585/275

(57) ABSTRACT

The invention relates to a process for selective hydrogenation by bringing a so-called C2 fraction feedstock into contact with a fixed catalyst bed, whereby said catalyst comprises a substrate and a metal phase that consists of either gold or palladium and gold with a molar ratio of gold to palladium of greater than 5, whereby the contact is carried out in the presence of a solvent that comprises at least one aromatic hydrocarbon, whereby the products that are obtained at the end of the selective hydrogenation can be separated from the solvent by distillation.

The invention relates to the field of selective hydrogenations and more particularly the development of a selective catalytic hydrogenation process of a C2 feedstock, for example a mixture of acetylene and ethylene, making it possible to improve the ethylene yields.

In the entire text the ethylene yield is such that:

Ethylene yield=100×[(ethylene mass leaving selective hydrogenation)/(ethylene mass entering selective hydrogenation)]

### PRIOR ART

The monoolefinic and diolefinic hydrocarbons that are produced by the thermal conversion processes such as steam-cracking are always associated with more greatly unsaturated hydrocarbons, in particular with acetylenic hydrocarbons. A selective hydrogenation of these latter hydrocarbons makes it possible to obtain the desired specifications for the monoolefinic and diolefinic hydrocarbons.

These hydrogenations are generally done with selective catalysts such as palladium on alumina catalysts. The current catalytic processes are not entirely satisfactory and the yields that are obtained can be improved in particular for the selective hydrogenation of the C2 fraction.

The C2 ethylene fraction that exits from a steam-cracking device generally has the following composition:

| | |
|---|---|
| Acetylene | 1 to 2% by volume |
| Ethylene | 70 to 90% by volume |
| Ethane | 10 to 30% by volume |

The acetylene content of the ethylene fraction should be brought to less than 2 ppm by volume by selective hydrogenation of the acetylene. This result is generally obtained by using isothermic or adiabatic processes that most often operate in the gaseous phase under approximately 2 to 3 MPa and at a temperature of usually between 60 and 150° C.

The drawbacks of these processes are numerous. The exothermicity of the reaction requires the installation of several reactors in a series because the gaseous phase does not promote the elimination of calories. In addition, the amount of catalyst that is used is large; a gaseous volumetric flow rate of 2,000 l/(l_{cata}·h) is common. The catalyst is not perfectly selective. The potential ethylene yields are 101 to 102% whereas the yields that are noted exceed 99.5% with difficulty. These low yields are due to two causes: an insufficient selectivity that leads to an excessive production of ethane and a parasitic polymerization of acetylene in more or less heavy products that are often called “green oils.” These polymers are deposited on the catalyst and greatly reduce the length of time of the cycles.

Processes that use a solvent have already been described for the hydrogenation of acetylene. This is the case of the process of the U.S. Pat. No. 4,128,595 in which it is recommended to use an inert hydrocarbon solvent. The use of a solvent offers several advantages relative to the process of hydrogenation in gaseous phase and in particular the following:
Better monitoring of the exothermicity of the reaction.
Improvement of the selectivity of the hydrogenation and therefore of the yield of the ethylene that is produced.
Improvement of the activity and the stability of the catalyst.

The process that is described in the Patent Application FR2552078 (A1) also consists in carrying out the selective hydrogenation of the acetylene in the presence of a catalyst with palladium in a liquid hydrocarbon solvent that contains 15 to 100% by weight of aromatic hydrocarbons, whereby said phase also contains an amino compound in solution.

### SUMMARY DESCRIPTION OF THE INVENTION

The invention relates to a selective hydrogenation process by bringing into contact a so-called C2 fraction feedstock with a fixed catalyst bed, whereby said catalyst comprises a substrate and a metal phase that consists either of gold or palladium and gold with a molar ratio of gold to palladiun of more than 5, whereby the contact is carried out in the presence of a solvent that comprises at least one aromatic hydrocarbon, whereby the products that are obtained at the end of the selective hydrogenation can be separated from the solvent by distillation.

### DETAILED DESCRIPTION OF THE INVENTION

The invention relates to a process for selective hydrogenation by bringing a feedstock that comprises at least C2 hydrocarbons into contact with a fixed catalyst bed in the presence of a solvent that comprises at least one aromatic hydrocarbon.

The feedstock that is named a C2 fraction generally comprises at least 80%, preferably at least 90%, by weight of hydrocarbons with 2 carbon atoms.

For example, the hydrocarbons with 2 carbon atoms that exit from a steam-cracking device generally have the following composition:

| | |
|---|---|
| Acetylene | 1 to 2% by volume |
| Ethylene | 70 to 90% by volume |
| Ethane | 10 to 30% by volume. |

The solvent can be a naphtha or kerosene fraction that comprises between 15 to 100% by weight of aromatic hydrocarbons.

The aromatic hydrocarbon is generally selected from the group that consists of benzene, toluene, ethylbenzene and xylene.

Aromatic hydrocarbon is preferably selected since it can be easily separated from the products of the hydrogenation. Preferably, the boiling point of the aromatic hydrocarbon is at least 10° C. greater, preferably 15° C. greater, than that of the feedstock.

The hydrogenation catalyst generally comprises gold and a substrate. The gold content is in general between 0.05 and 10% by weight, preferably between 0.1 and 5%, and even more preferably between 0.2 and 2% by weight.

The substrate can comprise at least one refractory oxide that is selected from the group that consists of oxides of magnesium, aluminum, silicon, zirconium, thorium, titanium, or cerium by themselves or mixed with one another. The substrate can also be a carbon, a silico-aluminate, a clay or any other compound that is known for being used as a substrate. Preferably, the substrate is an aluminum oxide (alumina) or silicon oxide (silica). Even more preferably, the substrate is alumina. The substrate can come in the form of balls, extrudates, trilobes or monoliths.

Preferably, the substrate has a specific surface area of between 5 and 300 m²/g, preferably between 10 and 150 m²/g.

The catalyst can also comprise palladium with contents that can be between 0.001 and 1% by weight, preferably between 0.005 and 0.5% by weight, and even more preferably between 0.01 and 0.2% by weight. In this case, the gold and the palladium are generally present in the catalyst in an Au/Pd molar ratio of more than 5, preferably between 6 and 30, and preferably between 8 and 12.

### Preparation of the Supported Au Catalysts

An aqueous solution that contains the quantity of gold precursor that is necessary for depositing the desired gold content on the substrate is prepared.

The salt of the gold precursor that is used has a degree of oxidation of the metal that is greater than 0 and is soluble in aqueous solution. The salt of the gold precursor can be, for example, a halide. It can be selected preferably from the group that consists of gold chlorides, such as gold trichloride, tetrachloroauric acid, sodium tetrachloroaurate or potassium tetrachloroaurate. Preferably, the precursor that is used is tetrachloroauric acid.

The concentration of gold in the solution is generally between 2.5·10⁻⁵ and 5·10⁻³ mol/liter, preferably between 5·10⁻⁵ and 2.5·10⁻³ mol/liter, and even more preferably between 10⁻⁴ and 10⁻³ mol/liter.

The concentration of gold in the solution is generally adjusted based on the mass of the substrate. The substrate is then added to this solution, and then the solution is next heated to a temperature of between 60 and 100° C. A urea solution is then prepared so as to obtain a urea/gold molar ratio that is preferably between 20 and 200. This solution is generally added, preferably drop by drop, to the solution that contains the substrate and the gold precursor, while being stirred.

Generally, the length of time of the addition is between 15 minutes and 4 hours, according to the concentration of gold in the solution. After urea is added, the suspension is generally stirred continuously, generally for a length of time of between 30 minutes and 24 hours, at a temperature that is generally between 50 and 100° C. The solid that contains the substrate and the gold is then generally recovered and then washed several times using a solvent, preferably with distilled water.

The catalyst is then generally dried, under vacuum, preferably in the absence of light and at a temperature of between 15 and 30° C. It is then generally activated by carrying out a reduction under hydrogen at a temperature of between 100 and 500° C.

### Preparation of Supported Au/Pd Catalysts

According to a preferred variant, an aqueous solution that contains the quantity of gold precursor and the quantity of palladium precursor that are necessary for depositing the desired gold and palladium contents on the substrate is prepared. The salt of the gold precursor that is used has a degree of oxidation of the metal that is greater than 0 and is soluble in aqueous solution. The salt of the gold precursor can be a halide. It can preferably be selected from the group that consists of the gold chlorides, such as tetrachloroauric gold trichloride, sodium tetrachloroaurate or potassium tetrachloroaurate. Preferably, the precursor that is used is tetrachloroauric acid.

The concentration of gold in the solution is between 2.5·10⁻⁵ and 5·10⁻³ mol/liter, preferably between 5·10⁻⁵ and 2.5·10⁻³ mol/liter, even more preferably between 10⁻⁴ and 10⁻³ mol/liter. The concentration in gold in the solution is generally adjusted based on the substrate mass.

The salt of the palladium precursor that is used has a degree of oxidation of the metal that is greater than 0 and is soluble in aqueous solution. The salt of the palladium precursor can be a halide, a hydroxide, a nitrate, a nitrite or a sulfate, a salt that combines a halide, a hydroxide, a nitrate, a nitrite or a sulfate of the metal with an alkaline compound, with an alkaline-earth, with an amino group or with an ammonia group.

More preferably, it can be selected from the group that consists of palladium chloride, palladium bromide, palladium iodide, potassium hexachloropalladate, ammonium hexachloropalladate, potassium tetrabromopalladate, potassium tetrachloropalladate, ammonium tetrachloropalladate, sodium hexachloropalladate, sodium tetrachloropalladate, palladium nitrate, palladium nitrite, diaminepalladium nitrite, palladium sulfate, tetraamine palladium nitrate, palladium dichlorodiamine, and palladium acetate. Preferably, the precursor of palladium is palladium dichloride. The palladium concentration in the solution is between 10⁻⁶ and 10⁻³ mol/liter, preferably between 10⁻⁵ and 2·10⁻⁴ mol/liter.

The substrate is then generally added to the solution, and then the solution is heated to a temperature of between 60 and 100° C. A urea solution is then prepared so as to obtain a urea/(gold+palladium) molar ratio that is preferably between 20 and 200. This solution is added, preferably drop by drop, to the solution that contains the substrate and the gold precursor, while being stirred. Generally, the length of time of the addition is between 15 minutes and 4 hours, depending on the gold concentration in the solution. After the urea is added, the suspension is stirred continuously, generally for a length of time of between 30 minutes and 24 hours at a temperature of between 50 and 100° C. The solid that contains the substrate and the gold is then generally recovered, and then washed several times using a solvent, preferably with distilled water.

The catalyst is then generally dried, under vacuum, preferably in the absence of light, and at a temperature of between 15 and 30° C. It is then generally activated by carrying out a reduction under hydrogen at a temperature of between 100 and 500° C.

### Operating Conditions of the Hydrogenation of the C2 Fraction

The operating conditions of the hydrogenation of the C2 fraction are generally as follows:
Volumetric flow rate (gas VVH [hourly volumetric flow rate]) expressed in terms of the volumetric flow of a gaseous C2 fraction at normal temperature and pressure per volume of catalyst (l/(l_{cata}·h)): 500 to 20,000 h⁻¹, preferably 1,000 to 10,000 h⁻¹.
Total pressure: 1 to 5 MPa, preferably 1.5 to 4 MPa,
Temperature: 10 to 200° C., preferably 20 to 150° C.

The hydrogen flow rate is effectively adjusted based on the acetylenic hydrocarbon content of the feedstock. It is expressed in terms of mols of hydrogen per mol of acetylenic hydrocarbons introduced into the reactor. This molar ratio is generally between 1 and 10 and preferably between 1 and 2.

The flow rate of solvent (hydrocarbon) is effectively regulated relative to the volume of catalyst. The volumetric flow rate (liquid VVH) is expressed in terms of the liquid volumetric flow rate per volume of catalyst. It is generally between 0.5 and 12 h⁻¹, preferably 1 and 10 h⁻¹.

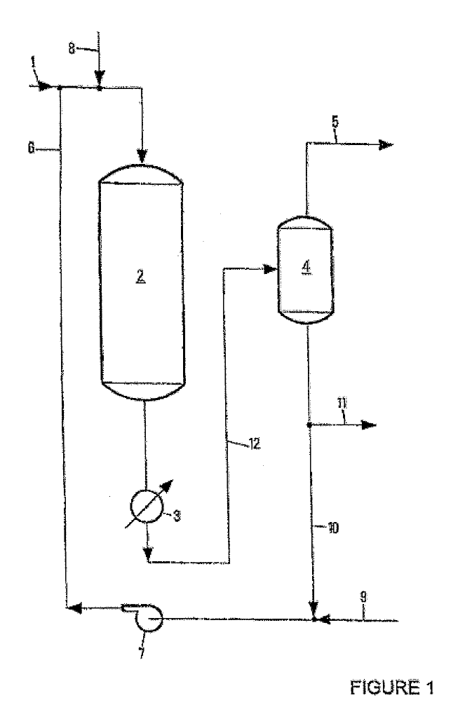

### Description of FIG. 1

The attached FIGURE exhibits an embodiment of the invention. In this example, the C2 fraction that is to be hydrogenated (1), hydrogen (8), and the liquid diluent (6) are introduced into the reactor (2). After cooling in the exchanger (3), the liquid-gas mixture is introduced via the line (12) into the separation flask (4) that separates the purified gaseous fraction (5) from the liquid diluent (10) that is recycled through the pump (7). The pipeline (11) makes it possible to purge a portion of the solvent, and the pipeline (9) makes it possible to add it.

### EXAMPLES

### Example 1

A gaseous fraction that contains 99% by weight of ethylene and 1% by weight of acetylene is treated.

The catalyst contains 0.2% by weight of palladium that is deposited on a substrate that consists of alumina with a specific surface area that is equal to 70 m2/g having a pore volume that is equal to 0.6 cm3/g. The substrate comes in the form of balls.

Before use, the catalyst is dried, under vacuum, in the absence of light and at a temperature of 20° C. It is then activated by carrying out a reduction under hydrogen at a temperature of 300° C.

The catalyst is arranged in a fixed bed in a tubular reactor.

The C2 fraction is passed into this reactor under the following conditions:

Gas VVH=2,500 h⁻¹

Pressure=2.5 MPa

Temperature=25° C.

H2/acetylene between 1.5 and 2 mols/mol

The H2/acetylene ratio is adjusted to obtain the desired conversion of the acetylene.

A solvent that also moves onto the catalytic bed at a flow rate that corresponds to liquid VVH=5 h⁻¹ is added to the C2 fraction. The solvent is collected in a separator flask and recycled as indicated in the diagram of FIG. 1. The solvent consists of 99.8% toluene and 0.2% by weight of piperidine.

Table 1 summarizes the results that are obtained after 5 hours of testing. The ethylene yields that are obtained per 2 ppm and 100 ppm of residual acetylene are indicated.

TABLE 1

| | Ethylene Yield | |
|---|---|---|
| | per 100 ppm | |
| | of Residual | Ethylene Yield per 2 ppm |
| Solvent | Acetylene | of Residual Acetylene |
| Toluene + 0.2% by | 100.72 | 100.69 |
| Weight of Piperidine | | |

### Example 2

A gold monometallic catalyst is prepared on an alumina substrate with a specific surface area of 70 m²·g⁻¹ and a pore volume that is equal to 0.6 cm³/g. The substrate comes in the form of balls.

The alumina substrate, in the form of balls, is immersed in a solution that contains HAuCl₄. The volume of solution that is used is 100 ml per gram of substrate. The concentration of Au in the solution is adjusted to deposit 1% by weight of gold. Then, this suspension is heated to 80° C.

A urea solution (10 ml per gram of substrate) is prepared so as to obtain a urea/gold molar ratio of 100. This solution is added drop by drop to the substrate suspension while being stirred energetically for one hour.

After adding urea, the suspension is stirred continuously for 16 hours, still at 80° C. The catalyst is then recovered by filtration. The balls are washed several times with distilled water.

The catalyst is then dried, under vacuum, in the absence of light and at a temperature of 20° C. It is then activated by carrying out a reduction under hydrogen at a temperature of 300° C.

In this example, the hydrogenation of a C2 fraction that contains 99% by weight of ethylene and 1% by weight of acetylene is carried out under the operating conditions of Example 1 by using the catalyst that is prepared above.

A solvent that also moves onto the catalytic bed at a flow rate that corresponds to the liquid VVH=5 h⁻¹ is added to the C2 fraction. The solvent is collected in a separator flask and recycled as indicated in the diagram of FIG. 1. The solvent consists of 99.8% toluene and 0.2% by weight of piperidine (Example 2a) or 100% toluene (Example 2b).

Table 2 summarizes the ethylene yields that are obtained for residual acetylene contents of 2 or 89 ppm.

TABLE 2

| | Example 2a (+ 0.2% by | Example 2b (without |
|---|---|---|
| Residual Acetylene | Weight of Piperidine): | Piperidine): |
| (ppm) | Ethylene Yield (%) | Ethylene Yield (%) |
| 89 | 101.62 | 101.63 |
| 2 | 101.51 | 101.52 |

The ethylene yield is improved relative to Example 1 since the ethylene yield per 2 ppm of residual acetylene changes from 100.69% to 101.5%.

### Example 3

A bimetallic palladium/gold catalyst is prepared on an alumina substrate with a specific surface area of 70 m²·g⁻¹ and a pore volume that is equal to 0.6 cm³/g. The substrate comes in the form of balls.

The alumina substrate is immersed in a solution that contains the metal precursors HAuCl₄ and PdCl₂. The volume of solution that is used is 100 ml per gram of substrate. The concentration of Au in the solution is adjusted to deposit 1% by weight of gold. The concentration of palladium in the solution is adjusted to deposit 0.054% palladium, or a gold/palladium molar ratio that is equal to 10. Then, this suspension is heated to 80° C.

A urea solution (10 ml per gram of substrate) is prepared so as to obtain a urea/(gold+palladium) molar ratio of 100. This solution is added drop by drop to the substrate suspension, while being stirred vigorously for one hour.

After urea is added, the suspension is stirred continuously for 16 hours, still at 80° C. The catalyst is then recovered by filtration. The balls are washed several times with distilled water.

The catalyst is then dried, under vacuum, in the absence of light and at a temperature of 20° C. It is then activated by carrying out a reduction under hydrogen at a temperature of 300° C.

The hydrogenation of a C2 fraction that contains 99% by weight of ethylene and 1% by weight of acetylene is carried out under the same operating conditions as the preceding examples 1 and 2.

A solvent that also moves onto the catalytic bed at a flow rate that corresponds to liquid VVH=5 h⁻¹ is added to the C2 fraction. The solvent is collected in a separator flask and recycled as indicated in the diagram of FIG. 1. The solvent consists of 99.8% toluene and 0.2% by weight of piperidine (Example 3a) or 100% toluene (Example 3b).

Table 3 summarizes the ethylene yield results obtained for residual acetylene contents from 2 to 1006 ppm.

TABLE 3

| | Example 3a (+0.2% by | Example 3b (without |
|---|---|---|
| Residual Acetylene | Weight of Piperidine): | Piperidine): |
| (ppm) | Ethylene Yield (%) | Ethylene Yield (%) |
| 1006 | 101.61 | 101.62 |
| 291 | 101.95 | 101.97 |
| 69 | 101.83 | 101.83 |
| 26 | 101.98 | 101.99 |
| 2 | 101.97 | 101.97 |

The ethylene yield is improved since the ethylene yield per 2 ppm of residual acetylene changes from 100.69% to 101.97% relative to the performance levels described in Example 1.

The preceding examples can be repeated with similar success by substituting the generically or specifically described reactants and/or operating conditions of this invention for those used in the preceding examples.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application Ser. No. 08/03.177, filed Jun. 6, 2008.

1. A process for selective hydrogenation by bringing a C2 fraction feedstock into contact with hydrogen and a catalyst, whereby said catalyst comprises a substrate that has a specific surface area of between 10 and 150 m²/g and a metal phase that consists essentially of either gold or a combination of palladium and gold with a molar ratio of gold to palladium of between 8 and 12, whereby the contact is carried out in the presence of a solvent that comprises at least one aromatic hydrocarbon, whereby resultant products obtained at the end of the selective hydrogenation can be separated from the solvent by distillation.

2. A process according to claim 1, in which the boiling point of the aromatic hydrocarbon(s) is at least 10° C. more than that of the feedstock.

3. A process according to claim 1, in which the solvent comprises between 15% and 100% by weight of aromatic hydrocarbons.

4. A process according to claim 1, the metal phase consists essentially of gold and, in which the gold content of the catalyst is between 0.05 and 10% by weight.

5. A process according to claim 1, the metal phase consists essentially said combination palladium and gold, and in which the palladium content of the catalyst is between 0.001 and 1% by weight.

6. A process according to claim 5, in which the gold/palladium molar ratio is between 6 and 30.

7. A process according to claim 1, in which the operating conditions of the hydrogenation of the C2 fraction are as follows:
volumetric flow rate expressed in terms of the volumetric flow of a gaseous C2 fraction at normal temperature and pressure per volume of catalyst is between 500 to 20,000 h⁻¹,
total pressure is between 1 to 5 MPa,
temperature is between 20 to 150° C.

8. A process according to claim 7, in which, in addition, the operating conditions of the hydrogenation of the C2 fraction are:
hydrogen flow rate is between 1 and 10 mol of hydrogen per mol of acetylenic hydrocarbons introduced into the reactor
solvent flow rate that is expressed in terms of the volumetric flow rate of liquid per volume of catalyst and per hour is between 1 and 10.

9. A process according to claim 4, wherein the gold content is between 0.1 and 5% by weight.

10. A process according to claim 4, wherein the gold content is between 0.2 and 2% by weight.

11. A process according to claim 3, wherein the aromatic hydrocarbon comprises at least one of benzene, toluene, ethylbenzene and xylene.

12. A process according to claim 5, in which the gold/palladium molar ratio is between 8 and 12.

13. A process according to claim 1, wherein the feedstock comprises ethylene and acetylene and the selective hydrogenation converts at least a portion of the acetylene into ethylene.

14. A process according to claim 13, in which the solvent comprises between 15% and 100% by weight of aromatic hydrocarbons.

15. A process according to claim 14, wherein the aromatic hydrocarbon comprises at least one of benzene, toluene, ethylbenzene and xylene.

16. A process according to claim 14, wherein the solvent is devoid of piperidine.

17. A process according to claim 13, wherein the solvent consists of aromatic hydrocarbons.

18. A process according to claim 17, wherein the aromatic hydrocarbon comprises at least one of benzene, toluene, ethylbenzene and xylene.
